(19) United States
(12) Patent Application Publication
(10) Pub. No.: US 2007/0027497 A1
(43) Pub. Date: Feb. 1, 2007

(54) Nerve stimulation for treatment of syncope

(75) Inventor: Steven M. Parnis, Pearland, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/190,398

(22) Filed: Jul. 27, 2005

(51) Int. Cl. A61N 1/00

(52) U.S. Cl. 607/45

(57) ABSTRACT

A method and apparatus for treating a syncope disorder is disclosed. The method comprises coupling a first electrode to a sympathetic nerve of the patient, and then applying a first therapeutic electrical signal to the electrode to stimulate the sympathetic nerve, wherein the stimulation causes an increase in the blood pressure of the patient to a level that is above a predetermined threshold level, whereby dizziness or fainting by the patient is deterred or prevented.

### CROSS-REFERENCE TO RELATED APPLICATIONS

None.

### STATEMENT REGARDING FEDERALLY SPONSORED RESEARCH OR DEVELOPMENT

Not applicable.

### BACKGROUND OF THE INVENTION

1. Field of the Invention

The present invention generally relates to methods and devices for treating syncope disorders in a mammal, and more particularly to methods that employ electrical neurostimulation of the sympathetic and/or parasympathetic nervous system to deter or prevent the occurrence of a vasovagal or orthostatic syncope episode.

2. Description of Related Art

Syncope, or fainting, is the partial or complete loss of consciousness with interruption of awareness of oneself and one's surroundings. It has been said that syncope accounts for one in 30 emergency room visits [1]. While it is rarely life threatening, and the dizziness or fainting episode usually resolves in a few minutes without treatment, syncope can in some cases severely diminish the quality of life for individuals who suffer recurring episodes. For instance, some people have difficulty getting out of bed in the morning without becoming dizzy or fainting. Those individuals are forced to arise gradually, lifting the head slowly and gradually assume sitting and standing positions over several minutes' time to compensate for gravitational effects that promote pooling of blood in the extremities. Medication is usually taken on a regular basis to keep blood pressure from falling too low.

Syncope is most commonly caused by conditions that do not directly involve the heart. Unexplained sudden episodes of syncope that occur as a result of inappropriate cardioinhibitory and vasodepressor responses, as may be provoked by head-up tilt or after assuming an upright posture, are usually termed “vasovagal syncope” or “dysautonomic syncope.” Vasovagal syncope is sometimes also referred to as neurocardiogenic or neurally mediated syncope, whereas dysautonomic syncope refers to a condition characterized by irregular neuroautonomic response during the body's attempt to maintain homeostasis. [2] Both types of syncope can occur in response to upright posture, such as orthostatic hypotension and postural orthostatic tachycardia syndrome (POTS). Hypotension (low blood pressure) may be primary, or it may be secondary to a condition such as tachyarrhythmia or bradyarrhythrmia Vasovagal and dysautonomic syncope have been described in the literature [3-4].

In patients with vasovagal syncope, the initial cardiovascular response to an upright posture may appear to be relatively normal. The syncope episode is due to an abrupt decrease in blood pressure, sometimes accompanied by a marked decrease in heart rate after a delayed period of head-up tilt, which triggers blood pooling in the lower extremities. The mean time to syncope in patients undergoing a tilt-table test is 25 minutes. According to U.S. Pat. No. 5,913,879, it has been postulated that vigorous contraction of a relatively empty ventricle activates myocardial sensory receptors, which, in susceptible persons, initiates an inhibitory reflex that results in hypotension, bradycardia or both. Vasodepressor reactions are believed to be caused by activation of unmyelinated left ventricular vagal nerve endings (i.e., C-fibers normally being excited by catecholamines, sympathetic nerve stimulation and left ventricular pressure. Spontaneous vasodepressor reactions often occur in the context of a sympathetic stimulation as a response to venous pooling. Thus, it is said that vasovagal syncope episode are preceded by venous pooling, a sudden heart rate increase, and vigorous ventricular contractions.

The dysautonomic syncope occurs in the presence of a failing or deficient autonomic system. In particular, sympathetic efferent activity is chronically impaired so that vasoconstriction is deficient. Patients with this condition are unable to compensate for an acute decrease in venous return, causing orthostatic hypotension (i.e., upon standing, blood pressure always falls). This failure of the autonomic nervous system to compensate for the postural change can occur immediately or can be delayed because of blood pooling in the lower extremities. The difference in mechanisms causing vasovagal syncope and dysautonomic syncope results in different treatment considerations. Bloomfield and his associates reviewed the pathophysiology of these two causes of syncope and developed an algorithm to guide diagnosis and treatment of vasovagal syncope and related disorders. Beta-adrenergic blocking agents (beta-blockers), fludrocortisone and midodrine are commonly used to treat patients with vasovagal syncope. Evidence of a dysautonomic response, in which the patient's blood pressure decreases without a significant increase in heart rate, suggests autonomic failure and is conventionally treated with fludrocortisone or midodrine [4].

A review of the apparent mechanisms involved in the development of neurally mediated syncope was carried out in 1993 by Rea and associates [5], who suggested that cardiac afferent neural activity may not be required for the development of vasodepressor responses in humans. Other potential mechanisms were said to include release of endogenous opioids or nitric oxide that may inhibit sympathetic nerve firing, and primary central nervous system activation that triggers cardioinhibitory and vasodepressor responses.

Pharmacologic treatments for alleviating vasovagal syncope have not proved to be highly effective. Pacemakers have also been investigated as a potential treatment, with mixed results [6-8]. Some reported substantial improvement over pharmacologic therapy in permanent pacemaker therapy, but less improvement in temporary pacing studies [6-7]. In a recent study, a statistically significant benefit was not found for pacemaker therapy for prevention of syncope in patients with vasovagal syncope [8]. U.S. Pat. No. 6,788,970 (Pacesetter, Inc.) discloses, with respect to vasovagal syncope, that there can be a delay before the pacemaker begins increasing the heart rate in response to detection of bradycardia. For many patients subject to recurrent vasovagal syncope, a significant drop in blood pressure occurs before the heart rate slows significantly. The drop in blood pressure causes significantly less blood filling the ventricles, and as a result there is simply not enough incoming blood to pump. Merely pumping the heart faster does not increase the overall blood pressure sufficiently to avoid cerebral hypoperfusion leading to transient unconsciousness. Therefore, the blood pressure will already have dropped to the point where the increase in heart rate is ineffective to avoid loss of consciousness by the time the pacemaker begins to increase the heart rate. For at least some patients subject to recurrent vasovagal syncope, it is possible that there would be no significant drop in heart rate, only a drop in blood pressure. Accordingly, a pacemaker programmed to prevent vasovagal syncope based solely upon detection of bradycardia will have little or no effect.

U.S. Pat. No. 6,885,888 (Rezai) suggests effecting physiological disorders by placing at least one stimulation electrode at a specific location along the sympathetic nerve chain, adjacent to or in communication with at least one ganglion. Adjusting parameters such as frequency or pulse width of the electronic stimulation is proposed to minimize adverse consequences and increase beneficial effects. A number of physical disorders are said to be treatable in this manner.

U.S. Patent Application Publication No. 2004/0249416 (Yun and Lee) suggests treating a condition caused by an abnormality in a subject's autonomic nervous system by electrically modulating at least a portion of the subject's autonomic nervous system to increase the parasympathetic activity/sympathetic ratio in a manner effective to treat the subject for the condition. A profusion of various conditions are mentioned as being at least partially manifested by abnormal balance of the sympathetic and parasympathetic functions of the autonomic nervous system.

There remains a need for an effective and practical therapy for reducing or preventing the occurrence of syncope episodes in persons who suffer from recurring syncopic episodes, especially when a chronic syncope disorder is not adequately responsive to traditional drug treatment.

### BRIEF SUMMARY OF THE INVENTION

The methods of the present invention overcome at least some of the failings of previous methods directed at controlling vasovagal or dysautonomic syncope by appropriately stimulating a sympathetic nerve such that the patient's blood pressure is increased above a predetermined lower threshold level which is sufficient to prevent syncope in the patient.

In accordance with certain embodiments of the present invention, a method is provided for treating syncope in a patient suffering from a syncope disorder. The method comprises coupling a first electrode to a sympathetic nerve of the patient; and applying a first therapeutic electrical signal to said electrode to stimulate said sympathetic nerve, wherein said stimulation causes an increase in the blood pressure of the patient to a level that is above a predetermined threshold level, whereby dizziness or fainting by the patient is deterred or prevented.

In some embodiments, sympathetic nerve stimulation is employed in concert with stimulation of the parasympathetic nervous system to deter or prevent syncope to facilitate maintaining a favorable balance between sympathetic and parasympathetic responses. The combined treatment is expected to benefit patients suffering from recurrent vasovagal or orthostatic syncope episodes. Accordingly, certain methods of treating an above-described syncope disorder also comprise coupling a second electrode to a vagus nerve of the patient; and applying a second therapeutic electrical signal to said second electrode to stimulate said vagus nerve, wherein said stimulation of said vagus nerve produces a physiological result selected from the group consisting of decreasing the blood pressure of the patient to a level that is above said predetermined threshold level, and counteracting an increase in blood pressure caused by said stimulation of said sympathetic nerve. In certain embodiments, counteracting said increase in blood pressure comprises maintaining the blood pressure of said patient between a predetermined lower threshold and a predetermined upper threshold by applying said first and second therapeutic electrical signals to said nerves sequentially, whereby dizziness or fainting by the patient is deterred or prevented. In certain embodiments, counteracting said increase in blood pressure comprises maintaining the blood pressure of said patient between a predetermined lower threshold and a predetermined upper threshold by applying said first and second therapeutic electrical signals to said nerves simultaneously, whereby dizziness or fainting by the patient is deterred or prevented. In certain embodiments, the stimulation is applied to a site in the cervical plexus, or to at least a portion of a nerve in the thoracic nerve chain, or to at least a portion of a nerve in the cardiac sympathetic chain, or to at least a portion of a nerve on the esophagus, or is applied to at least a portion of a splanchnic nerve.

Certain embodiments of the present invention provide a method of treating a syncope disorder that comprises providing an electrical signal generator capable of generating a pulsed electrical signal; providing at least one electrode; implanting the electrical signal generator in the patient's body; surgically coupling said first electrode to said sympathetic nerve of the patient; coupling the electrical signal generator to said first electrode; generating a predetermined pulsed electrical signal by said electrical signal generator; and applying said pulsed electrical signal to said first electrode to stimulate said sympathetic nerve, wherein said stimulation causes an increase in the blood pressure of the patient to a level that is above a predetermined threshold level, whereby dizziness or fainting by the patient is deterred or prevented.

In accordance with certain embodiments of the present invention a method of treating syncope is provided, the method comprising: providing an electrical signal generator capable of generating a pulsed electrical signal; providing at least one electrode; implanting the electrical signal generator in the patient's body; surgically coupling said first electrode to a sympathetic nerve of the patient; coupling the electrical signal generator to said electrode; generating a predetermined pulsed electrical signal by said electrical signal generator; and applying said pulsed electrical signal to said first electrode to stimulate said sympathetic nerve, whereby said stimulation causes an increase in blood pressure of the patient to a level that is above a predetermined threshold level, whereby fainting is deterred or prevented.

In certain embodiments, an above-described method the step of applying said pulsed electrical signal to said sympathetic nerve comprises increasing the magnitude of an existing pulsed electrical signal applied to said sympathetic nerve. In certain embodiments, the method comprises programming said electrical signal generator to define said pulsed electrical signal by a plurality of predetermined electrical parameters including a current magnitude, a pulse frequency, and a pulse width, wherein said defined electrical parameters are effective for deterring or preventing dizziness or fainting by the patient when said electrical signal is applied to said sympathetic nerve. In certain embodiments, a first therapeutic electrical signal comprises an acute electrical stimulation signal and a chronic electrical stimulation signal, and the method further comprises applying said acute electrical stimulation signal to said sympathetic nerve to alleviate an acute syncope episode; and applying said chronic stimulation signal to said sympathetic nerve to deter or prevent a recurrence of syncope. In certain embodiments, the first therapeutic electrical signal is defined by an on-time during which said signal is applied to said sympathetic nerve and an off-time during which no signal is applied to said nerve, said signal being effective to counteract an undesirable blood pressure increase resulting from parasympathetic stimulation. In some embodiments the electrical stimulation is delivered to a sympathetic nerve at programmed time intervals (e.g., on-time comprises about 30 seconds and said off-time comprises about five minutes) without regard to the physical condition of the patient, time of day, or other variables that might influence the need for, and/or efficacy of, the stimulation. This type of stimulation is referred to as “passive stimulation,” and is intended to reduce or eliminate recurrence of syncope episodes.

In other embodiments, the stimulus is delivered to the selected sympathetic nerve in response to detection of a physiological event or condition. Such responsive stimulation is referred to as “feedback” or “active stimulation,” and may be useful in treating nascent or ongoing syncope episodes. Accordingly, inclusion in a neurostimulation system of a capability for sensing a postural position makes it possible to automatically increase stimulation as needed to prevent syncope episodes. Accordingly, in certain embodiments of the present invention an above-described method of treating a syncope disorder also comprises providing a sensor (e.g., an accelerometer) to detect postural changes of said patient; coupling said sensor to said electrical signal generator; detecting a change in the patient's posture; and applying said pulsed electrical signal to said sympathetic nerve in response to said detected change in posture, whereby deterrence or prevention of dizziness or fainting is enhanced.

In certain embodiments, a method for treating syncope is provided which employs a sensor for detecting a change in a predetermined physiological parameter in the patient; coupling said sensor to said electrical signal generator; detecting a change in said predetermined physiological parameter; and applying said pulsed electrical signal to said sympathetic nerve in response to said detected change in said physiological parameter, whereby deterrence or prevention of dizziness or fainting is enhanced. In certain embodiments, the physiological parameter comprises a blood catecholamine level.

In certain embodiments, a method for treating syncope is provided which employs a sensor for detecting a manual activation command from the patient; coupling said manual activation sensor to said electrical signal generator; detecting a manual activation command by the patient experiencing a precursor symptom of syncope; and applying said pulsed electrical signal to said sympathetic nerve in response to said detected manual activation command, whereby deterrence or prevention of dizziness or fainting is enhanced.

In accordance with certain embodiments of the present invention an above-described method also comprises administering a pharmaceutical agent to said patient to lower blood pressure.

Certain embodiments of the present invention provide a method of deterring or preventing fainting in an individual suffering from a syncope disorder in which the method comprises stimulating the individual's sympathetic nervous system such that the individual's blood catecholamine level is increased, causing augmentation of vasomotor tone of the individual's vascular system and thereby deterring fainting. In some embodiments, the method comprises detecting a change in a physiological parameter that is indicative of syncope or pre-syncope; and applying a therapeutic electrical signal to a sympathetic nerve of the patient effective to cause an increase in blood catecholamine level in the patient, whereby vasomotor tone in said patient is augmented and fainting is deterred or prevented.

Also provided in accordance with certain embodiments of the present invention is an implantable apparatus for deterring or preventing fainting in a patient suffering from a syncope disorder, comprising a battery; a first electrode adapted for coupling to a sympathetic nerve of the patient; a programmable electrical signal generator coupled to said battery and adapted for applying a first therapeutic electrical signal to said first electrode to cause an increase in the blood pressure of the patient to a level that is above a predetermined threshold level, whereby dizziness or fainting by the patient is deterred or prevented. In some embodiments, the apparatus further comprises a second electrode adapted for coupling to a parasympathetic nerve of the patient, wherein said electrical signal generator is further adapted for applying a second therapeutic electrical signal to said second electrode to balance an effect of said first therapeutic electrical signal on the patient's autonomic nervous system.

Reduction or elimination of side-effects caused by pharmacologic therapy and prevention of syncope, as well as the advantage of providing on-demand stimulation as needed for acute treatment of syncope episodes is expected to be of particular benefit to patients. These and other embodiments, features and advantages will be apparent in the detailed description and drawings that follow.

### BRIEF DESCRIPTION OF THE DRAWINGS

FIG. 1 is a simplified partial front view of a patient illustrating one embodiment of a neurostimulator configuration for applying an electrical signal to a site in the patient's cervical plexus, in accordance with an embodiment of a treatment regimen of the present invention.

FIG. 2 is a front view of an implantable electrical signal generator as employed in the configuration of FIG. 1.

FIG. 3 illustrates a magnified view of a paddle-type electrode assembly for attachment to a nerve, according to the configuration of FIG. 1.

FIG. 4 is a simplified representation of a programmed output signal waveform as delivered to a sympathetic nerve, in accordance with certain embodiments of the present invention.

FIG. 5 is a flow chart illustrating a basic neurostimulator-implemented method of treating and controlling a chronic syncope disorder, according to an embodiment of the present invention.

### DETAILED DESCRIPTION OF PREFERRED EMBODIMENTS

Definitions.

Including and comprising are used in an open-ended fashion, and thus should be interpreted to mean “including, but not limited to.”

Couple or couples is intended to mean either an indirect or direct electrical connection. Thus, if a first device couples to a second device, that connection may be through a direct electrical connection, or through an indirect electrical connection via other devices and connections.

As used herein, the terms “stimulating” and “stimulator” generally refer to delivery of a signal, stimulus, or impulse to neural tissue for affecting neuronal activity of a neural tissue (e.g., a sympathetic nerve). The effect of such stimulation on neuronal activity is termed “modulation”; however, for simplicity the terms “stimulating” and “modulating,” and variants thereof, are sometimes used interchangeably herein. The effect of delivery of the signal to the neural tissue may be excitatory, inhibitory, or of a blocking nature, and may potentiate acute and/or long-term changes in neuronal activity. For example, the effect of “stimulating” or “modulating” a nerve may comprise one or more of the following effects: (a) changes in neural tissue to initiate an action potential (bi-directional or uni-directional), (b) inhibition of conduction of action potentials (endogenous or externally stimulated) or blocking (hyperpolarizing or collision blocking), (c) changes in neurotransmitter/neuromodulator release or uptake, receptors, gated ion channels or synapses which can be excitatory, inhibitory or of a blocking nature, and (d) changes in neuro-plasticity or neurogenesis of neural tissue.

Electrical stimulation signals of various types may be used in the present invention. Typical cranial nerve stimulation techniques involve providing a pulsed electrical signal in a series of discrete pulse bursts. A pulsed electrical signal is one in which flow of current during an on-time period is separated by short periods (typically milliseconds or seconds) of no current flow. A non-pulsed signal, as used herein, refers to a signal in which a current is always being delivered during the on-time. Pulses within a pulse burst are typically defined by a plurality of programmable parameters including a current magnitude (usually expressed in milliamps), a pulse width (usually expressed in microseconds or milliseconds), and a frequency (usually expressed in Hz). Pulse bursts are usually delivered according to the programmed pulse characteristics during a programmed on-time interval, and bursts are usually separated by defined off-time periods, in which no stimulation signal is provided. Although the invention is described with respect to pulsed electrical signals, non-pulsed signals may also be used. It should be noted that non-pulsed signals may be delivered according to a programmed or random on-time and off-time; however, unless the on-time periods have breaks in current flow within each on-time period, the signal remains a non-pulsed signal as used herein.

A continuous signal, as the term is used herein, refers to an electrical signal without a distinct on-time and off-time. Accordingly, conventional neurostimulation techniques are generally non-continuous signals. A continuous signal may be delivered as either a pulsed signal having a constant or random time period between pulses, as a non-pulsed signal with a constant or random time between stimulation signals, or as a purely continuous signal with no break in current flow at all (although other parameters, such as current magnitude and polarity, may vary within the signal).

Description

Applicants propose a method of providing a therapeutic electrical stimulation signal to a sympathetic nerve to treat syncope disorders, especially vasovagal syncope, by deterring or preventing syncope episodes or pre-syncope episodes (e.g., dizziness, fainting, lightheadedness, confusion, visual blurring). Treatment preferably comprises providing a therapeutic electrical signal in a manner to increase the patient's blood pressure above a fist threshold to ameliorate or avoid a syncope episode. Additional stimulation of the parasympathetic nerves, especially the left and/or right vagus nerve(s), may facilitate balance of the autonomic nervous system and assist in preventing syncope and/or avoid side effects associated with sympathetic nerve stimulation alone. Stimulation of the trigeminal nerve and the glossopharyngeal nerve is also expected to be similarly beneficial in establishing and maintaining balance of the parasympathetic/sympathetic nervous systems.

Neurostimulator

An exemplary neurostimulation system for treating syncope disorders comprises a neurostimulator which includes a signal generator, electrode and leads that, in preferred embodiments, are implanted in the body of the patient. The system also comprises an external unit for communication with the signal generator for, e.g., programming of the stimulation parameters by the attending physician according to the needs of the particular patient, and for monitoring the implanted device operation, through telemetry. For embodiments providing passive stimulation, the implanted signal generator may be programmed to deliver stimulation according to a programmed on-time and off-time, for intermittent stimulation that may be linked to the patient's circadian cycle, or for continuous stimulation. Alternatively, or additionally, the system may be designed to provide active or feedback stimulation, in which case a sensor is included and the electrical signal generator is able to be automatically triggered to deliver the prescribed therapy in response to sensing predetermined levels a body parameter indicating possible or actual occurrence of a syncope episode, e.g., low blood pressure, tachyarrhythmia, bradyarrhythmia, acute postural changes, or other parameters. Another system design for operation in active stimulation mode includes a sensing device for detecting a patient input signal, e.g., a magnet, to manually trigger stimulation as needed, for example, when the patient feels the onset of dizziness or other subjective pre-syncope condition.

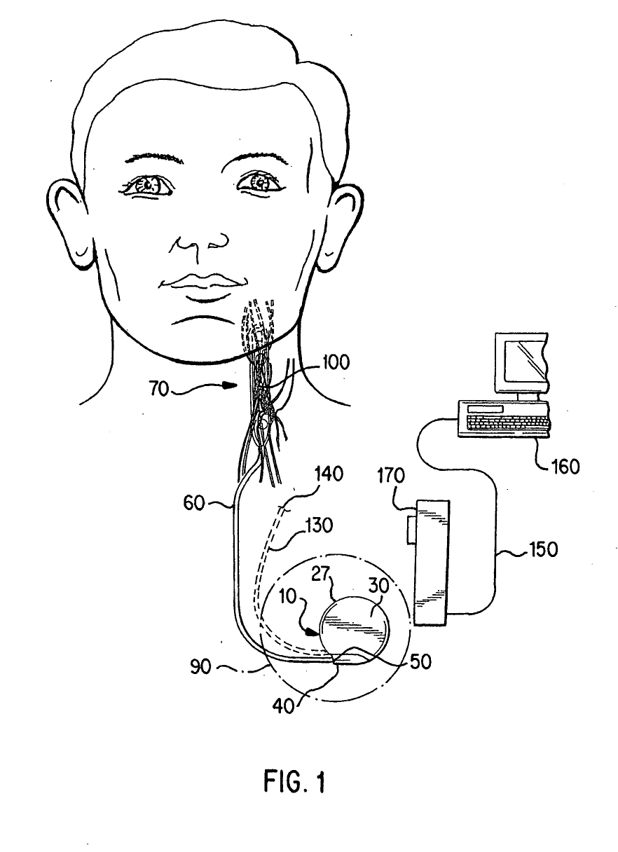

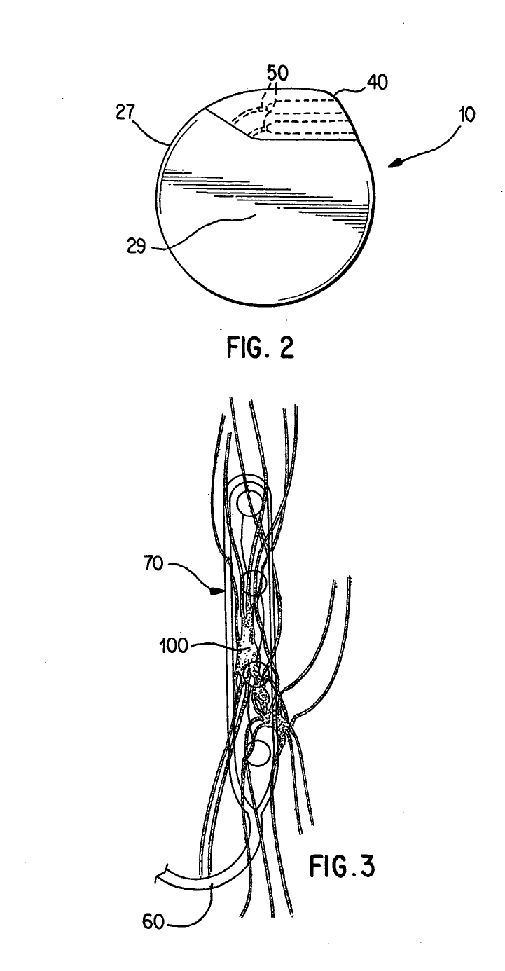

FIG. 1 illustrates a representative neurostimulation system (neurostimulator) 1 for selectively stimulating a sympathetic or parasympathetic nerve, such as cervical plexus 100 of a patient. A suitable apparatus for providing parasympathetic nerve stimulation, which may also be employed to provide sympathetic nerve stimulation, is described in U.S. Pat. No. 5,154,172 (Cyberonics, Inc), and the NeuroCybemetic Prosthesis or NCP™ device is available from Cyberonics, Inc., Houston, Tex., U.S.A. Alternatively, any other suitable form of neurostimulator may be employed. The preferred neurostimulator generally includes an electrical signal generator 10, at least one lead assembly (lead) 60, an electrode assembly 70, and an external programming system 150. Certain parameters of the electrical signals generated by the neurostimulator are programmable, preferably by means of an external programmer, in a conventional manner for implantable electrical medical devices. As also shown in FIG. 2, implantable electrical signal generator 10 is provided with a main body 30 comprising a case or shell 27 with a header 40 having at least one electrical connector 50 for connecting, respectively, to at least one lead 60. Case 27 houses a programmable electronics package (not shown).

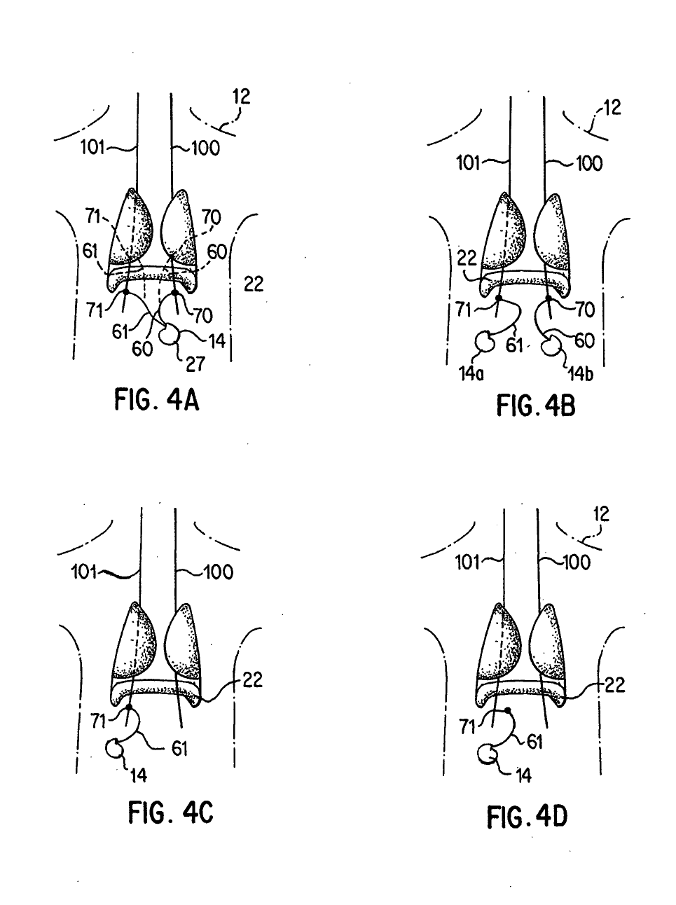

Referring to FIG. 3, a stimulating nerve electrode assembly 70, preferably comprising a paddle electrode, is conductively connected to the distal end of an insulated electrically conductive lead assembly 60. Lead wire in lead assembly 60 can be removably attached at its proximal end to a connector 50 on case 27. Electrode assembly 70 preferably comprises a paddle-type configuration and comprises a conductive material such platinum, iridium, platinum-iridium alloy, and/or an oxide of a foregoing material. The electrode assembly may include an anchoring tether for attaching to body tissue. Any suitable type of electrode may be employed that minimizes trauma to the neural tissue, such as cervical plexus 100, and which allows body fluid interchange with the stimulation site.

As shown in FIG. 1, external programming system 150 is preferably capable of wireless (e.g., radio frequency) communication with the electrical signal generator 10, and comprises a computer 160 and a wand 170 having an RF transmitter and receiver. Computer 160 preferably comprises a handheld computer operable by a healthcare provider. Wand 170 is capable of communicating with a receiver and transmitter in signal generator 10, and may be used to receive data from or transmit data to the electrical signal generator 10.

A variation of the above-described treatment configuration additionally provides for receiving and utilizing feedback from a sensed body parameter to the signal generator. Also shown in FIG. 1 (dashed line), the treatment assembly may further comprise a sensing lead 130 coupled at a proximal end to header 40 at connector 50. A sensor 140 is coupled to the distal end of sensing lead 130 (dashed line). Sensor 140 may comprise a temperature sensor, a blood parameter sensor, a heart parameter sensor, a brain parameter sensor, or other selected body parameter that is determined to be indicative of low blood pressure or other pre-syncope condition. The sensor may also comprise a nerve sensor for sensing activity on a nerve such as the sympathetic nerve, or on a parasympathetic nerve such as the vagus nerve.

Treating a Syncope Disorder

Vagus nerve stimulation alone is insufficient to alleviate vasovagal or dysautonomic syncope. The present method provides for electrically stimulating the sympathetic nervous system alone (primary stimulation), or in combination with simultaneous or sequential simulation of the parasympathetic nervous system (secondary stimulation), to treat chronic syncope disorders. Without wishing to be bound by a particular theory to explain the beneficial results obtained, it is believed that application of an appropriate therapeutic electrical signal interrupts C5 reactivation that can cause syncope. By appropriately stimulating the sympathetic nervous system instead of, or in addition to, the parasympathetic nervous system, activity of the patient's autonomic nervous system is maintained in a favorable relationship or balance, so that syncope is avoided. It is known that too much stimulation of one side of the autonomic nervous system (i.e., parasympathetic) can cause hypotension and vasodilation. Upon rising to a standing position, for instance, the parasympathetic nervous system is stimulated and dizziness or fainting results in some individuals.

Suitable sympathetic nerves for application of electrical stimuli to alleviate syncope disorders are located primarily in the neck, chest and abdomen. FIG. 1 illustrates a representative treatment system in which a stimulus electrode is coupled to a target location in the cervical plexus 100 in the neck of the patient. In another suitable alternative configuration an electrode is coupled to the thoracic sympathetic nerve chain or the cardiac sympathetic chain (not shown). Still other suitable sites for attachment of the electrode include a sympathetic nerve on the lower esophagus of the patient, preferably above the esophageal/stomach junction. Another suitable electrode placement site is a splanchnic nerve, which runs from the trunk of the sympathetic nerve, on each side of the spinal column, to the prevertebral ganglia which lie in front of the aorta.

A representative treatment protocol generally includes implanting a stimulus generator and one or more electrode assembly of a neurostimulator assembly into the patient to generate an electrical output signal, preferably configured as a sequence of pulses in which the electrical and timing parameters have programmable values. These parameter values are selected by the attending physician to be within ranges predetermined to be appropriate for the treatment. As an example, the appropriately configured pulse signal is applied to a sympathetic nerve such as the cervical plexus in the neck of the patient through an electrode set of a lead implanted on the nerve. The nerve stimulation is designed to modulate the electrical activity of the nerve and release neurotransmitters, serving as a neuromodulator to specifically cause a responsive increase in the patient's blood pressure sufficient to alleviate pre-syncope symptoms and prevent loss of consciousness by the patient, without increasing the blood pressure and heart rate above predetermined appropriate levels. Continued administration of one or more appropriately configured therapeutic electrical signal, either intermittent or sustained, is designed to help establish a balance between sympathetic and parasympathetic nervous activity in order to deter or prevent future reoccurrences of pre-syncopic or syncopic episodes.

As defined above, stimulating or modulating a nerve refers to delivery of an electrical stimulus to cause a responsive effect on the neural impulses traveling on the nerve. Predetermined therapeutic electrical signals are applied which result in beneficial effects that are excitatory, inhibitory or blocking in nature, or may potentiate other beneficial long-term or short-term effects. For instance, an electrical signal may be selected to provide exogenous (i.e., artificial) action potentials in one or more fibers of a nerve bundle, and/or may be selected to be incapable of generating an action potential and which are delivered for another purpose, such as blocking endogenous (i.e., native) action potentials from continuing on the nerve. Stimulating one or more sympathetic nerve may increase catecholamine levels in the blood, augmenting the vasomotor tone of the vascular system and thereby deterring syncope. The specific stimulating signal pattern used to achieve a desired effect of the sympathetic modulation for a prescribed treatment is selected based on various factors, including the individual patient, the specific nature of any underlying cardiovascular disorder or nervous disorder which may contribute to the patient's syncope episodes, and the nerve fibers to be activated The stimulation strategy also depends on factors such as whether a symptom or indicator of the patient's syncope disorder can be subjectively sensed for the purpose of manually activating the neurostimulator, or whether a physiologic parameter can be detected to trigger the stimulation, and whether a refractory period after the stimulation interval allows the benefits of the nerve activity modulation to persist.

Primary Stimulation of a Sympathetic Nerve. Referring again to the configuration shown in FIG. 1, a representative procedure for applying a primary therapeutic electrical signal to a patient's cardiac sympathetic chain (cervical plexus) in the neck, to produce a beneficial increase in blood pressure sufficient to deter or prevent syncope, employs the above-described neurostimulator system 1. The signal generator 10 is implanted in the patient's chest in a pocket or cavity formed by the implanting surgeon below the skin (indicated by a dashed line 90), at a suitable location as determined by the surgeon. For example, placement may be similar to a customary implantation procedure for a pacemaker pulse generator, or the signal generator may be implanted in the patient's abdominal region via a left laparotomy incision. The electrode assembly 70 is surgically coupled to the cervical plexus 100 in the patient's neck (FIG. 1). The paddle electrode is preferably configured so as to minimize the electrical stimulation threshold by providing for a relatively large stimulation contact area on the nerve. Lead assembly 60 is preferably secured in such a way as to retain the ability to flex with movement of the neck, such as by a suture connection 80 to nearby tissue. While the electrode 72 of electrode assembly 70 is shown in the preferred embodiment of directly contacting the nerve 100, it is to be understood that alternative electrode placements are also contemplated in which the electrode does not directly contact the nerve, provided that the electrode is electrically coupled to the nerve 100. Alternatively, instead of a paddle another suitable electrode design could be used.

Referring again to the representative treatment configuration illustrated in FIG. 1, neurostimulator 10 generates electrical stimuli in the form of electrical pulses according to one or more programmed parameters for stimulation of the sympathetic nerve. Typically, the stimulation parameters include pulse current, pulse width, frequency, and on-time or off-time. A table of ranges for each of these stimulation parameters is provided in Table 1. The magnitude of the various signal parameters (i.e., pulse current, pulse width, frequency, and on-time or off-time) are preferably optimized, and the operating range(s) is/are established (predetermined), at the time of surgical implantation of the device. The maximum amplitude of the current should be adjusted by the physician until an absence of undesirable effects associated with the stimulation is observed (e.g. such that the patient does not experience pain at the stimulation site when the stimulus is applied, or such that the heart rate does not change more than 10-20%.) Preferably a suitable safety margin is provided when establishing the permitted operating range. The initial therapeutic signal parameters are preferably customized by the physician at the time of surgical implantation by applying a first programmed signal to the electrodes, observing whether syncope can be provoked in a standard tilt-table test, and then observing the patient and making appropriate changes to the programmed instructions. A second signal comprising the resulting second set of parameters is then applied to the electrodes, and the patient is again observed during a repeated tilt-table test. The adjusting and testing steps are repeated as necessary until a satisfactory therapeutic stimulation signal is obtained which provides the desired decrease or elimination of syncope symptoms. The current of the final therapeutic stimulation signal will usually range from 0.5 to 3.0 mA, more typically about 1.5 mA is used. Since undesirable side effects of sympathetic nerve stimulation may change noticeably with time over a course of days after implantation, the current level is preferably checked after implantation, especially in the first few days after implantation and commencement of treatment, to determine whether any adjustment is necessary to maintain an effective regimen.

TABLE 1

| Parameter | Range | |
|---|---|---|
| Output Current | 0-6.0 | mA |
| Pulse Width | 1-1500 | μsec |
| Frequency | 0.5-250 | Hz |
| On-time | 1 sec-unlimited | |
| Off-time | 0 sec-unlimited | |
| Frequency Sweep | 10-100 | Hz |
| Random Frequency | 10-100 | Hz |

On-time and off-time parameters are used to define an intermittent pattern in which a repeating series of pulses is generated for stimulating the nerve during the on-time (such a sequence is referred to as a “pulse burst”), followed by a period in which no pulses are generated, and the nerve is allowed to recover or rest from the stimulation provided by the pulse burst. The on/off duty cycle of these alternating periods of stimulation and no stimulation preferably has a ratio in which the off time may be set to zero, providing continuous stimulation, or may be as long as one day or more, in which case the stimulation is provided once per day or at even longer intervals. Typically, however, the ratio of the off-time to on-time ranges from approximately 0.5 to 10, i.e., the off-time is from half as long as the on-time to ten times the length of the on-time. One suitable pattern of stimulation of the cervical plexus comprises a non-continuous pulsed electrical signal having an on-time of about 30 seconds and an off-time of about five minutes, for 24 hours a day, 7 days a week A treatment regimen such as this is expected to keep the blood pressure above a predetermined lower threshold so that a syncope episode does not occur.

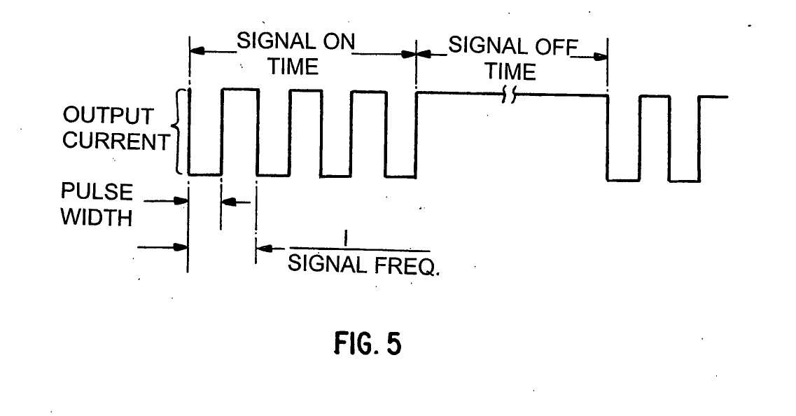

FIG. 4 is a simplified representation of the output signal waveform delivered by signal generator 10 to electrode assembly 70, which illustrates the configurable (programmable parameters on-time, off-time, frequency, pulse width, and output current for the output signal. Nominally, the width of each pulse is set to a value not greater than about 1 millisecond, more typically 250-500 microseconds, and the pulse repetition frequency is programmed to be in a range of about 20 to 250 Hz. A steady frequency of about 30 Hz may be used, although a non-uniform frequency may prove to be more advantageous in treating a particular patient for a particular syncope disorder. Frequency is altered during a pulse burst by either a frequency sweep from a low frequency to a higher frequency or vice versa. Alternatively, the timing between adjacent individual pulses within a burst is randomly changed.

It is contemplated that application of two or more different stages or phases of such therapeutic electrical signals will be especially advantageous for managing acute episodes of syncope and for long-term deterrence or prevention of recurrent episodes in patients (chronic syncope). For instance, a first, acute stage of treatment employs a first therapeutic electrical signal having a first set of predetermined parameters for quickly adjusting the patient's blood pressure to a level that is within a prescribed range (in which syncope is unlikely to occur). A second, chronic stage signal then follows, which is designed to provide a continuous lower level of stimulation, having a second set of predetermined parameters for deterring or preventing repeat episodes of syncope. The electrical and timing parameters of the stimulating signal used for sympathetic nerve stimulation (SNS) described herein will be understood to be merely illustrative and not as constituting limitations on the scope of the invention, except insofar as recited in the claims. U.S. patent application Ser. No. 11/046,430 [Docket No. 1000.065] (Cyberonics, Inc.), entitled “Multi-Phase Signal for Stimulation by an Implantable Device,” discloses a method and apparatus for providing a suitable multi-phasic stimulation regimen.

Although the use of an implanted stimulator is preferred, treatment may instead be administered using an external stimulator with an internally implanted lead and electrode that are inductively coupled to the external stimulator. Wholly external stimulation may also be used on an out-patient basis, although implantation of at least one electrode and one lead is preferred for a more efficient electrical coupling to the nerve. Moreover, implantation of one or more neurostimulators advantageously allows the patient to be completely ambulatory during treatment, so that normal daily routine activities are unaffected.

Still another activation modality for stimulation is to program the output of the neurostimulator to the maximum amplitude which the patient can tolerate, with cycling on and off for a predetermined period of time followed by a relatively long interval without stimulation. When sympathetic nerve stimulation is provided based solely on programmed off-times and on-times (which may also be used to provide stimulation according to circadian rhythms), the stimulation is referred to as passive, inactive, or non-feedback stimulation.

Active or Feedback Loop Stimulation of a Sympathetic Nerve. In contrast to the above-described passive stimulation mode, in some treatment regimes a therapeutic electrical signal is triggered by one or more feedback loops according to a predetermined change within the patient's body or in response to a predetermined action by the patient. This mode of treatment is referred to as active or feedback loop stimulation. When a sensing capability is provided, the electrical signal generator and/or external processor is/are additionally configured for measuring, sensing, recording, monitoring the physiological activity, physiological event, and/or physiological threshold. This is accomplished by detecting and evaluating a postural change or a variation in catecholamine level in the blood, for instance. The processor and controller are configured such that a predetermined therapeutic electrical signal is applied in response to detection by the system of one or more physiological parameters which is considered to be indicative of syncope or pre-syncope.

Manually Activated Mode. The most common form of feedback loop stimulation is manually triggered stimulation, in which the patient manually causes the activation of a stimulation pulse burst outside of the programmed on-time/off-time cycle. For example, if the patient feels the onset of pre-syncope, he or she may manually activate the neurostimulator to stimulate the sympathetic nerve, thereby causing a responsive elevation of blood pressure sufficient to stop a syncope or pre-syncope symptom and sufficient to keep the patient from fainting. Subject to approval by the physician, and appropriate programming, the patient may be given some limited control over the therapy. For instance, the patient may be allowed to alter the signal frequency, current, duty cycle, or a combination of those parameters, within prescribed ranges. If indicated, the neurostimulator may be programmed to generate and sustain a predetermined stimulus for a relatively long period of time in response to manual activation. In this way, the treatment stimulates the nerve continuously or intermittently such that a patient's blood pressure does not drop below a predetermined lower threshold level whereby recurrence of syncope is deterred or prevented.

Patient activation of the neurostimulator may involve use of an external control magnet for operating a reed switch in the implanted device, for example. Certain other applicable techniques of manual and automatic activation of implantable medical devices are disclosed in U.S. Pat. No. 5,304,206 (Cyberonics, Inc.). For example, means for manually activating or deactivating the stimulus generator may include a sensor such as a piezoelectric element mounted to the inner surface of the generator case and adapted to detect light taps by the patient on the implant site. One or more taps applied in fast sequence to the skin above the location of the stimulus generator in the patient's body may be programmed into the device as the signal for activation of the generator, whereas two taps spaced apart by a slightly longer time gap is programmed as the signal for deactivation, for example. The therapy regimen performed by the implanted device(s) remains that which has been pre-programmed by means of the external programmer, according to the prescription of the patient's physician in concert with recommended programming techniques provided by the device manufacturer. In this way, the patient is given limited but convenient control over the device operation, to an extent that is determined by the program dictated and/or entered by the attending physician. The patient also may activate the neurostimulator using other suitable techniques and/or apparatus. When passive stimulation is combined with active stimulation, as needed, a steady state stimulation pattern is established, with the patient being able to supplement or boost the level of stimulation within a prescribed range, according to the perceived symptoms of pre-syncope.

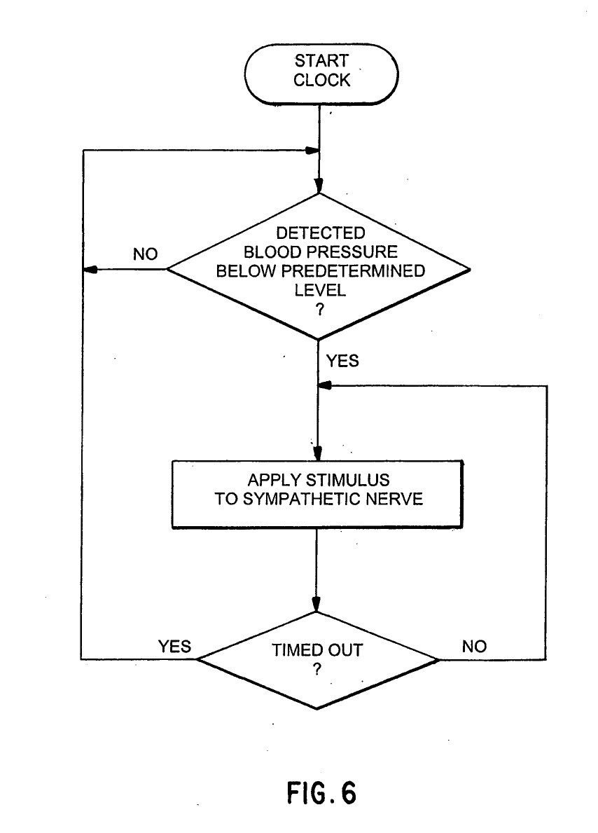

Physiological Sensing Mode. Reference is now made to the feedback-enhanced configuration shown in FIG. 1, with the sensing assembly depicted in dashed lines. In instances in which physiological feedback-enhanced stimulation is desired, without manually triggering by the patient, sensor 140 preferably senses a body parameter that corresponds to a symptom or physical indication of syncope or pre-syncope (e.g., manifested by a sudden decrease in blood pressure, and/or a sudden increase in heart rate). Sensing of catecholamine levels in the patient's blood may be suggestive of vascular tone. If sense electrodes are to be utilized to detect early indicators or onset of syncope, a signal analysis circuit is preferably incorporated in the neurostimulator for processing and analyzing the signals from the sensor 140. FIG. 5 is a flow chart illustrating a basic neurostimulator-implemented method of treating and controlling a chronic syncope disorder. The system continually monitors the blood pressure of the patient (systolic and/or diastolic) to detect a sudden drop in blood pressure. Upon detection of one or more selected syncope or pre-syncope indicator, the processed digital signal is supplied to a microprocessor in the neurostimulator device, to trigger application of the stimulating signal to the selected sympathetic nerve. For instance, a pressure that is below a preset lower threshold level is taken as indicating a need to initiate treatment to alleviate pre-syncope or syncope. If, however, the patient's blood pressure as monitored by the system is above the predetermined threshold level, no further stimuli are applied to the nerve. The applicable thresholds and stimulation parameters are determined for patients on an individual basis. The system may remain in monitoring mode. In a contemplated variation of this feedback-enhanced treatment regime, the detection of a syncope indicator may be used to trigger a stimulation program comprising different stimulation parameters from a passive stimulation program, such as having a higher current or a higher ratio of on-time to off-time.

Although the preferred location for application of a therapeutic electrical signal to a sympathetic nerve is the cervical plexus, it is expected that other sympathetic nerves may be stimulated at sites other than, or in addition to, the cervical plexus, and that such stimulation will also produce at least some alleviation or deterrence of syncope. For instance, the cardiac sympathetic chain, the thoracic sympathetic chain, a sympathetic nerve on the lower esophagus of the patient, preferably above the esophageal/stomach junction, or a splanchnic nerve, which runs from the trunk of the sympathetic nerve, on each side of the spinal column, to the prevertebral ganglia which lie in front of the aorta.

Secondary Stimulation of a Parasympathetic Nerve. In some instances, it will be desirable to not only stimulate the sympathetic nervous system, but to also stimulate the parasympathetic nervous system in order to balance the interplay of synergistic and antagonistic activities of the autonomic nervous system, particularly for treating syncope disorders. A representative example of this dual mode of stimulation includes coupling a second therapeutic electrical signal to a vagus nerve of the patient and applying to the nerve a second predetermined sequence of electrical pulses generated by the same or a different electrical signal generator. Secondary stimulation is applied at a suitable site on the main vagus nerve or the left and/or right vagus nerve, at a neck, near-diaphragmatic, or other suitable location. Both supra-diaphragmatic and sub-diaphragmatic locations are encompassed within the term “near-diaphragmatic,” and where the latter term is used either location is intended.

Although the preferred embodiments provide for secondary stimulation of the vagus nerve, preferably the left and/or right vagus nerve, it is expected that other cranial nerves may be stimulated instead of, or in addition to, to the vagus nerve to produce at least some alleviation or deterrence of syncope. For instance, secondary stimulation of the trigeminal nerve (5th cranial nerve) and the glossopharyngeal nerve (9th cranial nerve) is expected to provide beneficial results when applied in concert with primary stimulation of a sympathetic nerve. Without further elaboration, it is believed that one skilled in the art can, using the description herein, utilize the present invention to its fullest extent. The foregoing embodiments are to be construed as illustrative, and not as constraining the remainder of the disclosure in any way whatsoever. It will be understood that a device such as that described herein is generally required to be approved or sanctioned by government authority for marketing as a medical device implantable in a patient together with electrode means to treat a syncope disorder by electrically stimulating a sympathetic nerve, with or without secondary stimulation of a parasympathetic nerve of the patient.

While the preferred embodiments of the invention have been shown and described, modifications thereof can be made by one skilled in the art without departing from the spirit and teachings of the invention. Many variations and modifications of the invention disclosed herein are possible and are within the scope of the invention. Accordingly, the scope of protection is not limited by the description set out above, but is only limited by the claims which follow, that scope including all equivalents of the subject matter of the claims. The disclosures of all patents, patent applications and publications cited herein are hereby incorporated herein by reference, to the extent that they provide exemplary, procedural or other details supplementary to those set forth herein.

### REFERENCES

The following publications are cited by number in the foregoing text:
1. MedicineNet http://www.medterms.com (Aug. 2, 2003).
2. Sealey, B and Lui K. “Diagnosis and management of vasovagal syncope and dysautonomia,” AACN Clin Issues. July-September 2004; 15(3):462-477.
3. Kaufnaun, H. “Neurally mediated syncope and syncope due to autonomic failure: differences and similarities,” J. Clin Neurophysiol. May 1997; 14(3):183-196.
4. Bloomfield, D M et al., “Putting it together: a new treatment algorithm for vasovagal syncope and related disorders,” Am J Cardiol. Oct. 21, 1999; 84(8A):33Q-39Q.
5. Rea, R and Thames, M D. “Neural control mechanisms and vasovagal syncope,” J. Cardiovasc Electrophysiol. October 1993; 4(5):587-595.
6. Sheldon, R. “Role of pacing in the treatment of vasovagal syncope,” Am J Cardiol. Oct 21, 1999; 84(8A):26Q-32Q.
7. Raj, S R and Sheldon R S. “Role of pacemakers in treating neurocardiogenic syncope,” Curr Opin Cardiol. January 2003; 18(1):47-52.
8. Connolly, S J et al., “Pacemaker therapy for prevention of syncope in patients with recurrent severe vasovagal syncope,” JAMA May 7, 2003; 289(17):2224-2229.

What is claimed is:

1. A method of treating syncope comprising:
coupling a first electrode to a sympathetic nerve of a patient suffering from a syncope disorder; and
applying a first therapeutic electrical signal to said electrode to stimulate said sympathetic nerve, wherein said stimulation causes an increase in the blood pressure of the patient to a level that is above a predetermined threshold level, whereby dizziness or fainting by the patient is deterred or prevented.

2. The method of claim 1 further comprising:
coupling a second electrode to a vagus nerve of the patient; and
applying a second therapeutic electrical signal to said second electrode to stimulate said vagus nerve, wherein said stimulation of said vagus nerve produces a physiological result selected from the group consisting of decreasing the blood pressure of the patient to a level that is above said predetermined threshold level, and counteracting an increase in blood pressure caused by said stimulation of said sympathetic nerve.

3. The method of claim 2 wherein counteracting said increase in blood pressure comprises maintaining the blood pressure of said patient between a predetermined lower threshold and a predetermined upper threshold by applying said first and second therapeutic electrical signals to said nerves sequentially, whereby dizziness or fainting by the patient is deterred or prevented.

4. The method of claim 2 wherein counteracting said increase in blood pressure comprises maintaining the blood pressure of said patient between a predetermined lower threshold and a predetermined upper threshold by applying said first and second therapeutic electrical signals to said nerves simultaneously, whereby dizziness or fainting by the patient is deterred or prevented.

5. The method of claim 1 wherein said sympathetic nerve comprises a target location in the cervical plexus.

6. The method of claim 1 wherein said sympathetic nerve comprises at least a portion of a nerve on the esophagus.

7. The method of claim 1 comprising:
providing an electrical signal generator capable of generating a pulsed electrical signal;
providing at least one electrode;
implanting the electrical signal generator in the patient's body;
surgically coupling said first electrode to said sympathetic nerve of the patient;
coupling the electrical signal generator to said first electrode;
generating a predetermined pulsed electrical signal by said electrical signal generator; and
applying said pulsed electrical signal to said first electrode to stimulate said sympathetic nerve, wherein said stimulation causes an increase in the blood pressure of the patient to a level that is above a predetermined threshold level, whereby dizziness or fainting by the patient is deterred or prevented.

8. The method of claim 7 comprising:
providing a sensor to detect postural changes of said patient;
coupling said sensor to said electrical signal generator;
detecting a change in the patient's posture; and
applying said pulsed electrical signal to said sympathetic nerve in response to said detected change in posture, whereby deterrence or prevention of dizziness or fainting is enhanced.

9. The method of claim 8 wherein said sensor is an accelerometer.

10. The method of claim 8 wherein applying said pulsed electrical signal to said sympathetic nerve comprises increasing the magnitude of an existing pulsed electrical signal applied to said sympathetic nerve.

11. The method of claim 7 comprising:
providing a sensor for detecting a change in a predetermined physiological parameter in the patient;
coupling said sensor to said electrical signal generator;
detecting a change in said predetermined physiological parameter; and
applying said pulsed electrical signal to said sympathetic nerve in response to said detected change in said physiological parameter, whereby deterrence or prevention of dizziness or fainting is enhanced.

12. The method of claim 11 wherein said physiological parameter comprises a catecholamine level measured in the patient's blood.

13. The method of claim 7 comprising:
providing a sensor for detecting a manual activation command from the patient;
coupling said manual activation sensor to said electrical signal generator;
detecting a manual activation command by the patient experiencing a precursor symptom of syncope; and
applying said pulsed electrical signal to said sympathetic nerve in response to said detected manual activation command, whereby deterrence or prevention of dizziness or fainting is enhanced.

14. The method of claim 7 comprising:
programming said electrical signal generator to define said pulsed electrical signal by a plurality of predetermined electrical parameters including a current magnitude, a pulse frequency, and a pulse width, wherein said defined electrical parameters are effective for deterring or preventing dizziness or fainting by the patient when said electrical signal is applied to said sympathetic nerve.

15. The method of claim 1, wherein said first therapeutic electrical signal comprises an acute electrical stimulation signal and a chronic electrical stimulation signal, said method further comprising:
applying said acute electrical stimulation signal to said sympathetic nerve to alleviate an acute syncope episode; and
applying said chronic stimulation signal to said sympathetic nerve to deter or prevent a recurrence of syncope.

16. The method of claim 1, wherein said first therapeutic electrical signal is defined by an on-time during which said signal is applied to said sympathetic nerve and an off-time during which no signal is applied to said nerve, said signal being effective to counteract an undesirable blood pressure increase resulting from parasympathetic stimulation.

17. The method of claim 16, wherein said on-time comprises about 30 seconds on and said off-time comprises about five minutes.

18. The method of claim 1 further comprising administering a pharmaceutical agent to said patient to lower blood pressure.

19. A method of treating syncope comprising:
providing an electrical signal generator capable of generating a pulsed electrical signal;
providing at least one electrode;
implanting the electrical signal generator in the body of a patient suffering from a syncope disorder;
surgically coupling said first electrode to a sympathetic nerve of the patient;
coupling the electrical signal generator to said electrode;
generating a predetermined pulsed electrical signal by said electrical signal generator; and
applying said pulsed electrical signal to said first electrode to stimulate said sympathetic nerve, whereby said stimulation causes an increase in blood pressure of the patient to a level that is above a predetermined threshold level, whereby the syncope disorder is ameliorated.

20. A method of treating syncope comprising:
stimulating the sympathetic nervous system of an individual suffering from a syncope disorder such that the individual's blood catecholamine level is increased, causing augmentation of vasomotor tone of the individual's vascular system and thereby deterring fainting.

21. The method of claim 20 comprising:
detecting a change in a physiological parameter that is indicative of syncope or pre-syncope; and
applying a therapeutic electrical signal to a sympathetic nerve of the patient effective to cause an increase in blood catecholamine level in the patient, whereby vasomotor tone in said patient is augmented and fainting is deterred or prevented.

22. The method of claim 21 wherein said stimulating comprises electrically stimulating a cervical plexus.
